(19) United States
(12) Patent Application Publication
(10) Pub. No.: US 2013/0006035 A1
(43) Pub. Date: Jan. 3, 2013

(54) RADIOTHERAPY PHANTOM

(75) Inventors: Adam H. AITKENHEAD, Stockport (GB); Ranald Mackay, Manchester (GB); Carl G. Rowbottom, Cheshire (GB)

(73) Assignee: The Christie NHS Foundation Trust, Manchester (GB)

(21) Appl. No.: 13/535,088

(22) Filed: Jun. 27, 2012

Related U.S. Application Data
(63) Continuation-in-part of application No. 13/529,222, filed on Jun. 21, 2012.
(60) Provisional application No. 61/499,728, filed on Jun. 22, 2011.

(51) Int. Cl. G01D 18/00; A61N 5/10; G01T 1/17

(52) U.S. Cl. 600/1; 378/207; 250/395

(57) ABSTRACT

The present invention relates to a phantom for use in the auditing or verification of a proposed radiation therapy regime for administration to a patient. The phantom comprises a housing which is shaped to simulate the anatomical shape of a human head and neck; and a radiation detector module configured to receive at least one radiation detector. The housing defines a cavity in which the radiation detector module can be removeably received such that the radiation detector module occupies a predetermined location within the simulated head and neck of the housing. Said predetermined location encompasses areas of the housing which simulate a target site to which it is proposed to administer radiation to the patient and a location of at least one organ that is susceptible to harm by administration of said radiation.

### CROSS-REFERENCE TO RELATED APPLICATIONS

This application is a continuation-in-part of U.S. application Ser. No. 13/529,222, filed Jun. 21, 2012, which claims the benefit under 35 U.S.C. §119(e) of U.S. Provisional Patent Application No. 61/499,728 filed Jun. 22, 2011, the disclosure of which is hereby incorporated herein by reference in its entirety.

### STATEMENT REGARDING FEDERALLY SPONSORED RESEARCH OR DEVELOPMENT

Not Applicable.

### BACKGROUND

The present invention relates to a radiotherapy phantom. Methods for auditing and verifying radiotherapy treatment regimes using the phantom are also described.

Radiotherapy or radiation therapy involves the use of ionising radiation in medicine. It can be used to control malignant cells in cancer treatment. It can also be used in a number of non-malignant conditions and in preparing the body for bone marrow transplantation.

In cancer treatment, radiotherapy may be the primary modality or may be an adjuvant to other modalities, such as surgery, chemotherapy, hormone therapy and/or immunotherapy. Commonly, the ionising radiation is applied to a target volume including the cancerous tumour and surrounding tissue. Radiation may also be applied to other areas of the body, such as draining lymph nodes involved with the tumour.

To minimise the risk of the radiotherapy harming healthy tissue sophisticated methods have been developed in which multiple beams of ionising radiation are directed towards the target volume from different positions around the volume. In this way, the dose of radiation incident upon the target volume is greater than that upon the surrounding tissue. Intensity Modulated Radiotherapy (IMRT) has been developed to further limit the harm to healthy tissue by allowing the intensity of the ionising radiation to be controlled so that the shape of the radiation beam can be matched to the shape of the tumour as closely as possible.

The risk of serious side-effects from radiotherapy is particularly high in tumours adjacent to the spinal cord. It is therefore especially important in cancers where a tumour is located within the head or neck region that the radiotherapy regime is carefully planed and verified before the ionising radiation is administered to the patient.

Pre-treatment verification of IMRT typically involves delivery of a predetermined radiotherapy treatment plan to a device containing a series of radiation detectors, known as a ‘phantom’, followed by a comparison of the measured dose against that predicted by a treatment planning system (TPS).

Existing verification methods can be split into two classes: (i) measurement of the fluence from a linear accelerator head using a two-dimensional (2D) array of detectors; and (ii) measurement of the dose distribution within an anatomical or semi-anatomical phantom. The first method using a 2D array of detectors is relatively quick, but is merely a check of the delivery system rather than a check of the combined dose distribution. It is also an incomplete test of the planning model because it fails to simulate the impact of physical inhomogeneities. The second method using a phantom provides a check of the delivered dose distribution as well as providing a more complete test of the planning model by including semi-anatomical or anatomical structures and/or inhomogeneities.

A number of different types of phantom are currently available. Most have detectors housed within a cylindrical casing which lacks an anatomically accurate shape. More recently, anatomically accurate phantoms have been developed but they are still generally limited by the fact that they are designed for use with a single type of detector, such as a diode, ionisation chamber, film, thermoluminescent dosimeter (TLD) or, more recently, a radio-sensitive gel.

### SUMMARY

Herein disclosed is a phantom for use in the auditing or verification of a proposed radiation therapy regime for administration to a patient, the phantom comprising:
a. a housing which is shaped to simulate the anatomical shape of a human head and neck; and
b. a radiation detector module configured to receive at least one radiation detector, wherein the housing defines a cavity in which the radiation detector module can be removeably received such that the radiation detector module occupies a predetermined location within the simulated head and neck of the housing, said predetermined location encompassing areas of the housing which simulate a target site to which it is proposed to administer radiation to the patient and a location of at least one organ that is susceptible to harm by administration of said radiation.

In an embodiment, the organ is the spinal cord. In an embodiment, the predetermined location encompasses areas of the housing which simulate target sites that are commonly selected for the administration of radiation to treat a cancer selected from the group consisting of nasopharynx, oropharynx, hypopharynx, tongue, tonsil, thyroid and neck.

In an embodiment, the cavity is dimensioned to encompass a majority of the volume of the simulated neck of the phantom. In an embodiment, the cavity is dimensioned to encompass areas of the simulated head of the phantom which simulate the pharynx and sinus cavities.

In an embodiment, the cavity defines a cylinder that tapers linearly from a first end to an opposite second end. In an embodiment, a diameter of the first end is around 1 to 20% larger than a diameter of the second end.

In an embodiment, the said cavity possesses a longitudinal length that is greater than a diameter of its ends. In an embodiment, the cavity possesses a longitudinal length that is 50 to 250% greater than a diameter of its ends. In an embodiment, the cavity defines a longitudinal axis that is inclined relative to the horizontal when the phantom occupies a typical radiotherapy treatment position.

In an embodiment, the cavity defined by the housing is adapted to receive one or more radiation detector modules supporting different types of radiation detectors. In an embodiment, each of the different types of radiation detector is selected from the group consisting of a radiosensitive gel, a radiosensitive film, a radiosensitive diode, an ionisation chamber, a thermoluminescent dosimeter, and a radioluminescent detector.

In an embodiment, the cavity defined by the housing is adapted to receive one or more radiation detector modules supporting different numbers of radiation detectors. In an embodiment, the cavity defined by the housing is adapted to receive one or more radiation detector modules supporting different arrangements of radiation detectors. In an embodiment, the radiation detector module defines a plurality of locations for receipt of a radiation detector.

In an embodiment, the plurality of locations are provided in an accurate path extending from a periphery of the radiation detector module to a centre of the radiation detector module. In an embodiment, the radiation detector is an ionisation chamber radiation detector.

In an embodiment, the radiation detector defines a rectangular slot extending across a diameter of the detector module and into the detector module for receipt of a sheet of radiosensitive film. In another embodiment, an outer surface of the radiation detector module defines at least one straight edge to co-operate with a complementary straight edge defined by the phantom to locate the radiation detector in a predetermined orientation relative to the phantom.

In an embodiment, the housing defines at least one cavity for receipt of a removable fixture at a location corresponding to that of a heterogeneity in the structure of at least one of the human head and the human neck. In an embodiment, the heterogeneity is an air cavity or a mandible. In an embodiment, the removable fixture is made of a different material to the remainder of the housing.

Herein also disclosed is a method for auditing a radiotherapy regime using a phantom of this disclosure. The method comprises:
a. creating a radiotherapy treatment plan on a patient CT dataset;
b. transferring said radiotherapy treatment plan from the patient CT dataset on to a radiotherapy phantom CT dataset;
c. recalculating a dose distribution within the phantom as required to ensure that a location of a radiotherapy dose will lie in substantially the same region of the phantom CT dataset as in the patient CT dataset; and
d. exporting the radiotherapy treatment plan to a radiotherapy treatment machine for delivery to the phantom.

In a further embodiment, the method further comprises exporting the radiotherapy treatment plan to a radiotherapy treatment machine for delivery to the patient.

Further disclosed herein is a method for verifying a proposed radiotherapy regime using a phantom of this disclosure. The method comprises:
a. selecting one or more detector locations within the phantom to be used to measure a predetermined delivered dose of radiation;
b. providing the phantom in a treatment position;
c. inserting a detector or a plurality of detectors into the phantom so that they occupy said pre-selected detector location or locations;
d. providing required inhomogeneities within the phantom;
e. delivering said predetermined dose of radiation to the phantom;
f. measuring the dose of radiation delivered to the phantom using the one or more detectors;
g. comparing the measured dose of radiation to the predetermined dose of radiation; and
h. determining any differences between the measured dose of radiation and the predetermined dose of radiation.

### DETAILED DESCRIPTION

An object of the present invention is to obviate or mitigate one or more of the aforementioned problems with current radiotherapy phantoms.

According to a first aspect of the present invention there is provided a phantom for use in the auditing or verification of a proposed radiation therapy regime for administration to a patient, the phantom comprising: a) housing which is shaped to simulate the anatomical shape of a human head and neck; and b) radiation detector module configured to receive at least one radiation detector, wherein the housing defines a cavity in which the radiation detector module can be removeably received such that the radiation detector module occupies a predetermined location within the simulated head and neck of the housing, said predetermined location encompassing areas of the housing which simulate a target site to which it is proposed to administer radiation to the patient and a location of at least one organ that is susceptible to harm by administration of said radiation.

The present invention therefore provides a radiotherapy phantom which is anatomically similar to a treatment site and which can be used with a range of different detectors.

The present invention provides a phantom for use in the auditing or verification of a proposed radiation therapy regime for administration to a patient, the phantom comprising: a) a housing which simulates the anatomical shape of a human head and neck; and b) a radiation detector module configured to receive at least one radiation detector, wherein the housing defines a cavity within the simulated head and neck of the housing in which the radiation detector module can be removeably received such that the radiation detector module occupies a predetermined location within the housing, said predetermined location encompassing a first area of the housing which simulates a target site to which it is proposed to administer radiation to the patient and said predetermined location encompassing a second area of the housing which simulates the location of at least one organ that is susceptible to harm by administration of said radiation.

The at least one organ may be any organ of the body that is at risk of being harmed by exposure to radiation. The organ is preferably the spinal cord since it is important that exposure of this organ to radiation during radiotherapy is accurately monitored so that the potential for damage is minimised.

It is preferred that the predetermined location within the housing which is occupied by the radiation detector module encompasses areas of the housing which simulate target sites that are commonly selected for the administration of radiation to treat cancers of the head and/or neck, including nasopharynx, oropharynx, hypopharynx, tongue, tonsil, thyroid and neck cancer. As explained in more detail below, the devisors of the present invention took X-ray computed tomography (CT) scan datasets from a plurality of patients lying in the standard radiotherapy treatment position and calculated average geometries for the external body, the mandible, the sinuses and the spinal cord. A phantom was then constructed which was based on those average geometries. A range of typical radiotherapy treatment plans for cancers of the head and neck was then mapped on to the phantom to identify typical planning target volume (PTV) locations. The location of at-risk organs within the head and neck region were also mapped on to the phantom. A cavity was formed in the phantom for receipt of a radiation detector module. The cavity was formed at a location within the phantom so that, upon receipt of the detector module, the module occupies a volume which encompasses the majority of the PTV locations and the at-risk organs mapped on to the phantom.

The cavity may be defined so as to occupy a volume which encompasses the majority of the PTV locations and the at-risk organs, with the detector module being of a size and shape so as to substantially fill the cavity.

Alternatively, the cavity may be larger than the detector module such that the module does not substantially fill the cavity, but rather leaves some space within the cavity unoccupied. Such space may be left unoccupied during use of the phantom, or it may be filled or partially filled by a further component containing no detectors, or it may be filled or partially filled by at least one further radiation detector module. That is, the cavity in the housing of the phantom may be configured so that it can receive two or more radiation detector modules, which may contain different types of detector, different numbers of detectors and/or different arrangements of detector.

The cavity is preferably dimensioned to cover a majority (i.e. greater than 50%), more preferably most, of the volume of the simulated neck region of the phantom and/or is preferably dimensioned so as to extend into the simulated head region of the phantom to also cover the pharynx and nasal/sinus cavities.

The cavity may take any appropriate shape. It preferably takes the general form of a cylinder, more preferably a cylinder that tapers regularly or linearly from one of its ends to its opposite end. The cavity is preferably in the form of a frustocone.

In a first preferred embodiment, a diameter of a first end of the cavity is larger than a diameter of a second end of the cavity which is opposite to said first end. The first end may be nearer to the head region of the phantom than the second end and the second end may be nearer to the neck region of the phantom than the first end, or vice versa.

The diameter of the first end of the cylinder may be around 1 to 20% larger than the diameter of the second end. More preferably the diameter of the first end is around 2 to 10% larger than the diameter of the second end. Still more preferably the first end diameter is around 4 to 8% larger than that of the second end, and most preferably the first end diameter is around 6% larger than that of the second end.

The cavity may have a longitudinal length that is greater than a diameter of either of its ends. Preferably, the length of the cavity is greater than both of its ends. The length of the cavity may be around 50 to 250% greater than the diameter of the cavity at its first and/or second end. More preferably the cavity's length is around 100 to 200% greater than its diameter at its first and/or second end. Yet more preferably the length of the cavity is around 125 to 175% greater than the diameter of the first and/or second end of the cavity and most preferably the length of the cavity is around 150 to 170% greater than the diameter of the first and/or second end of the cavity.

In the first preferred embodiment in which the diameter of the first end of the cavity is larger than that of the second end of the cavity, the length of the cavity is preferably around 130 to 170% greater than the diameter of the first end of the cavity and the length of the cavity is preferably around 140 to 180% greater that the diameter of the second end of the cavity. More preferably the length of the cavity is around 140 to 160% greater, most preferably around 150% greater, than the diameter of the first end of the cavity and the length of the cavity is around 150 to 170% greater, most preferably around 165% greater, than the diameter of the second end of the cavity.

The phantom is preferably designed such that longitudinal axis of the cavity will be inclined relative to the horizontal when the phantom occupies a typical radiotherapy treatment position, e.g. when simulating a patient lying on a couch with the back of the head and shoulders resting on the couch. The extent to which the longitudinal axis is inclined to the horizontal may be chosen to suit a particular application. That is, it may be chosen so as to ensure that the desired PTV locations and at-risk-organ locations are encompassed by the cavity while taking into account the size and shape of the cavity.

With the phantom occupying a typical radiotherapy treatment position it is preferred that the longitudinal axis of the cavity is inclined relative to the horizontal at an angle of around 10 to 40°, more preferably around 15 to 30°, or around 20 to 25°. Most preferably the longitudinal axis of the cavity lies at an angle of about 23.5° to the horizontal when the phantom occupies a typical radiotherapy treatment position.

In a particularly preferred embodiment of the phantom according to the present invention the cavity is in the shape of a linearly tapered cylinder having a longitudinal length of 24 cm, a diameter at the end nearer the head of the phantom of 9.5 cm, a diameter of the end nearer the neck of 9 cm, and whose longitudinal axis is inclined by 23.5° to the horizontal when the phantom occupies a standard radiotherapy treatment position.

The (or each) radiation detector module is preferably adapted to be able to support different types of radiation detectors. The different types of radiation detector that can be supported by a radiation detector module according to the present invention include a radiosensitive gel, a radiosensitive film, a radiosensitive diode, an ionisation chamber, a thermoluminescent dosimeter, and a radioluminescent dosimeter.

When using a radiosensitive film (e.g. an X-ray sensitive film) in the phantom it is possible to measure and analyse the delivered dose in a 2D plane. The radiation detector may define a rectangular slot extending across a diameter of the detector module and into the detector module for receipt of a sheet of radiosensitive film. As described in more detail below with reference to FIG. 5, the radiosensitive film may be positioned in a detector module and aligned such that the longitudinal axis of the simulated spinal cord lies in the plane of the film. An advantage of the present invention over prior art phantoms is that the detector module for the radiosensitive film can be easily rotated through a plurality of predetermined positions to allow the orientation of the film to be adjusted to suit the radiotherapy treatment plan under test. An outer surface of said radiation detector module may define at least one straight edge or a plurality of straight edges to co-operate with one or more complementary straight edges defined by the phantom. In this way, the radiation detector can be located in one or more predetermined orientations relative to the phantom. It will be appreciated that straight edges represent a simple and convenient means of defining a series of predetermined relative orientations for the detector module and phantom however any appropriate series of co-operating features may be defined by the detector module and phantom to achieve essentially the same result.

It is preferred that the (or each) radiation detector module can support different numbers of radiation detectors of the same type or of different types. While it may be preferred in some applications to use a single radiation detector within the or each module received in the phantom housing, in other applications it will be preferred to use two or more radiation detectors so that dosimetry measurements can be obtained at multiple locations during use of the phantom. All of these locations may be within a predetermined PTV for the type of cancer being treated. Alternatively, all of these locations may be within an area occupied by an at-risk organ or group of at-risk organs, or, as a further alternative some of these locations may be within the PTV and others within an area or areas occupied by an at-risk organ or organs. A detector module may be configured to receive any appropriate number of detectors. For example, it may be preferred to provide a detector module with three, four, five or more cavities, apertures, recesses or the like which are adapted to receive detectors. By way of further examples, the detector module may accommodate up to around 20 to 25 detectors, around 2 to 15 detectors or around 6 to 12 detectors.

The (or each) detector module may be configured to support different arrangements of radiation detectors of the same type or of different types. Multiple detectors may be arranged within a detector module in any desirable arrangement. By way of example, a plurality of detectors may be supported within a detector module in a linear or non-linear two-dimensional or three-dimensional array. Detectors may be arranged in curved, arcuate or circular arrangements within a detector. In a preferred embodiment the radiation detector module defines a plurality of locations for receipt of detectors, said predefined locations preferably being provided in an arcuate path extending from a periphery of the radiation detector module to a centre of the radiation detector module. Any desirable number of such locations may be provided, for example, around 8 to 16 may be appropriate, more preferably around 10 to 14, and most preferably around 12 location. In a first preferred embodiment said radiation detector module defines 12 locations for receipt of one or more ionisation chamber radiation detectors.

The flexibility of the phantom housing and detector module(s) in being able to accommodate such a wide range of detector types, number and arrangement represents a significant improvement as compared to prior art radiotherapy phantoms. Prior art phantoms have typically lacked either an anatomically faithful shape or the ability to support different types, numbers or arrangements of radiation detector.

Designing the phantom of the present invention to be able to receive one or more radiation detector modules occupying a location within both the simulated head and neck of the housing also represents a step forward in this technical field because it enables the user to monitor the levels of radiation being applied to both the head and neck region during a single radiotherapy test cycle rather than being limited to just the head and then having to carry out a second test in relation to the neck region, or vice versa, as is the case with prior art phantoms.

Additionally, by virtue of the detector module(s) occupying a location within the phantom which encompasses a simulated radiotherapy target site on the patient and a location of at least one at-risk organ, for example the spinal cord, affords the user with a more complete view of the effect that a planned radiotherapy regime is likely to have on a patient than many prior art phantoms in which detectors can only be positioned within a much smaller volume of the simulated head or neck region.

It is preferred that the phantom housing defines at least one cavity, which may be left empty or may receive at least one removable fixture, block or the like at one or more locations corresponding to heterogeneities in the structure of the human head and/or neck. The cavity in the housing may be left empty, such that the heterogeneity is an air cavity simulating the sinuses, or the cavity may be filled or partially filled with a block or fixture with a density similar to bone to simulate a mandible. It is preferred that said removable fixture is made of a different material to the remainder of the housing. While the housing may be formed from any suitable material, it is preferably formed from acrylonitrile butadiene styrene (ABS). The removable fixture may be formed from any appropriate material, such as a polyurethane foam or a polycarbonate.

A second aspect of the present invention provides a method for auditing a radiotherapy regime using a phantom according to the above-defined aspect of the present invention. The method comprises the creation of a treatment plan on a previously obtained patient CT dataset following the standard radiotherapy planning procedures. The treatment plan is then transferred from the patient CT dataset on to the phantom CT dataset and the dose distribution within the phantom recalculated as appropriate. The radiotherapy treatment plan is transferred from the patient CT dataset to the phantom CT dataset such that the location of the delivered dose lies in the same region of the phantom CT dataset as it does in the patient CT dataset. Once this has been achieved the radiotherapy treatment plan can then be exported to a radiotherapy treatment machine for delivery to the phantom, with measurement of the delivered dose preferably being made in the phantom using suitable detectors. The radiotherapy treatment plan may then be exported to a radiotherapy treatment machine for delivery to the patient.

A third aspect of the present invention provides a method for verifying a proposed radiotherapy regime using a phantom according to the first aspect of the present invention, the method comprising:
a. selecting one or more detector locations within the phantom to be used to measure a predetermined delivered dose of radiation;
b. providing the phantom in a treatment position;
c. inserting a detector or a plurality of detectors into the phantom so that they occupy said pre-selected detector location or locations;
d. providing required inhomogeneities within the phantom;
e. delivering said predetermined dose of radiation to the phantom;
f. measuring the dose of radiation delivered to the phantom using the one or more detectors;
g. comparing the measured dose of radiation to the predetermined dose of radiation; and
h. determining any differences between the measured dose of radiation and the predetermined dose of radiation.

With regard to step a., selecting one or more detector locations within the phantom to be used to measure a delivered dose of radiation, it is preferred that:
i. if a large array of detectors or a multipoint detector is available, the dose at as many detector locations as possible is measured and used for comparison to the radiotherapy treatment plan via a suitable analysis method, such as a gamma analysis (e.g. as described in Low et al. “A technique for the quantitative evaluation of dose distributions.” Med. Phys. 25(5) p. 656-661, 1998); alternatively,
ii. if a single detector or a single point detector is to be used, suitable measurement locations in the PTV and cord are identified. This may be achieved using computer code which identifies suitable points within regions such as the PTV and cord, with points being selected to lie in regions of low dose gradient where possible.

With regard to step b., setting up the phantom in the treatment position, it is preferred that this involves positioning the phantom on a treatment couch as a patient would be positioned. The phantom can be set up in the correct position by aligning scribe lines on the phantom with positioning lasers that are usually provided in area or room in which the radiotherapy will be administered to the patient. If required, positional offsets can be applied to ensure that the phantom occupies a position that is close as possible to that which the intended patient will occupy during treatment.

In step d., the inhomogeneities that are provided within the phantom are preferably the mandible and/or air cavity corresponding to the sinuses.

The comparison and determining of any differences between measured and predetermined doses preferably involves a comparison of the absolute dose values and, preferably, a comparison based upon a gamma analysis of the measured and predetermined doses.

An embodiment of the present invention will now be described, by way of example only, with reference to the accompanying drawings in which:

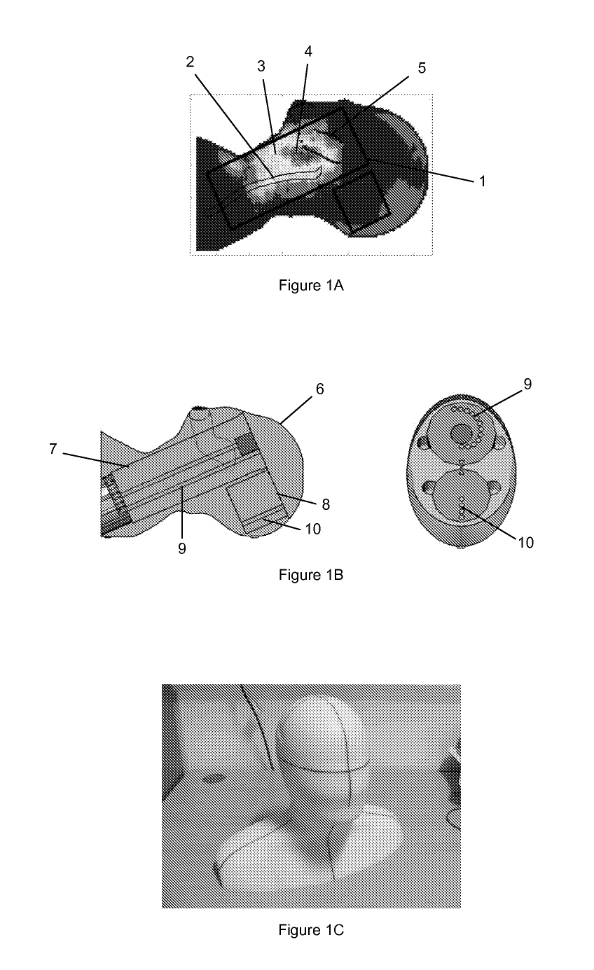

FIG. 1A is a colour-coded image of the PTV locations of 25 typical head and neck radiotherapy treatment regimes in the treatment of nasopharynx, oropharynx, hypopharynx, tongue, tonsil, thyroid and neck cancer. Sites that are more lightly coloured are generally more commonly targeted than those of a darker colour, save for the dark region in the centre of the light area which denotes the area most commonly included in a PTV.

FIG. 1B is a cross sectional image of a phantom according to the present invention including a detector module designed for receipt of a PinPoint™ionisation chamber radiation detector;

FIG. 1C is a photograph of a computer numerical control (CNC) machined prototype phantom according to the present invention formed using ABS (ρ=1.05 gcm⁻³);

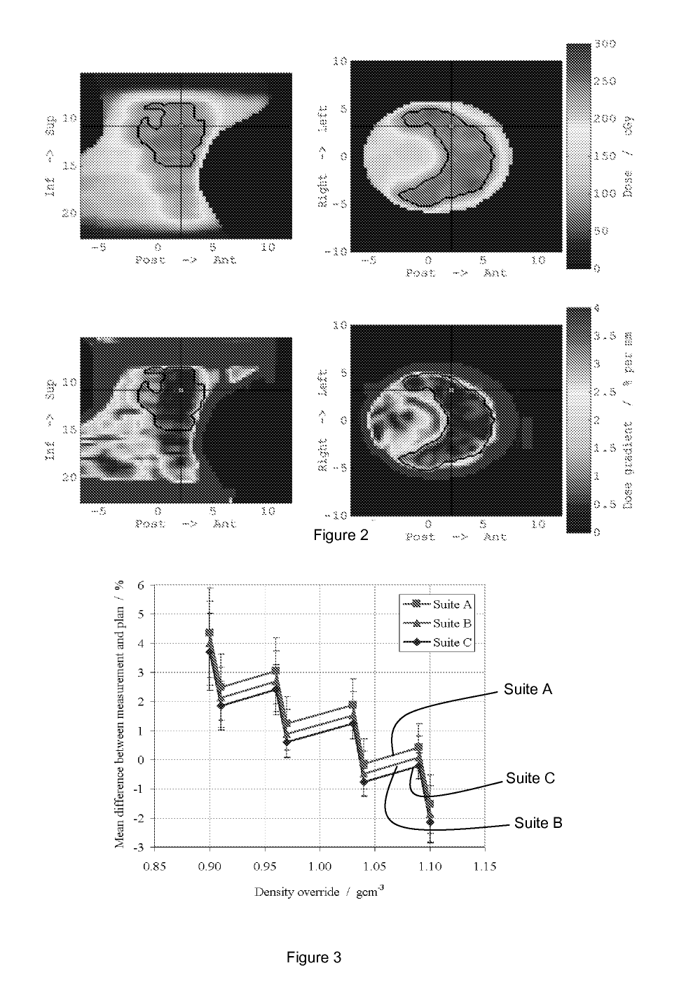

FIG. 2 are colour coded images of the overall dose (upper images) and the dose gradient (lower images) in relation to a selected measurement point (where the cross-hairs intersect) mapped on to the geometry of a phantom according to the present invention. The PTV used is that for a typical thyroid radiotherapy plan. The maximum dose gradient within any individual treatment beam at the selected point is 1.35%/mm, and the dose gradient for all beams combined is 0.34%/mm;

FIG. 3 is a plot of the mean differences between measured and planned doses for 6 simple (10×10 cm square) beams and 6 different detector locations. Planned dose distributions were computed in Pinnacle v9.0 using bulk density overrides. Measurements were made using a phantom according to the present invention in its homogeneous configuration and a PinPoint™ionisation chamber radiation detector (active volume 0.015 cm³);

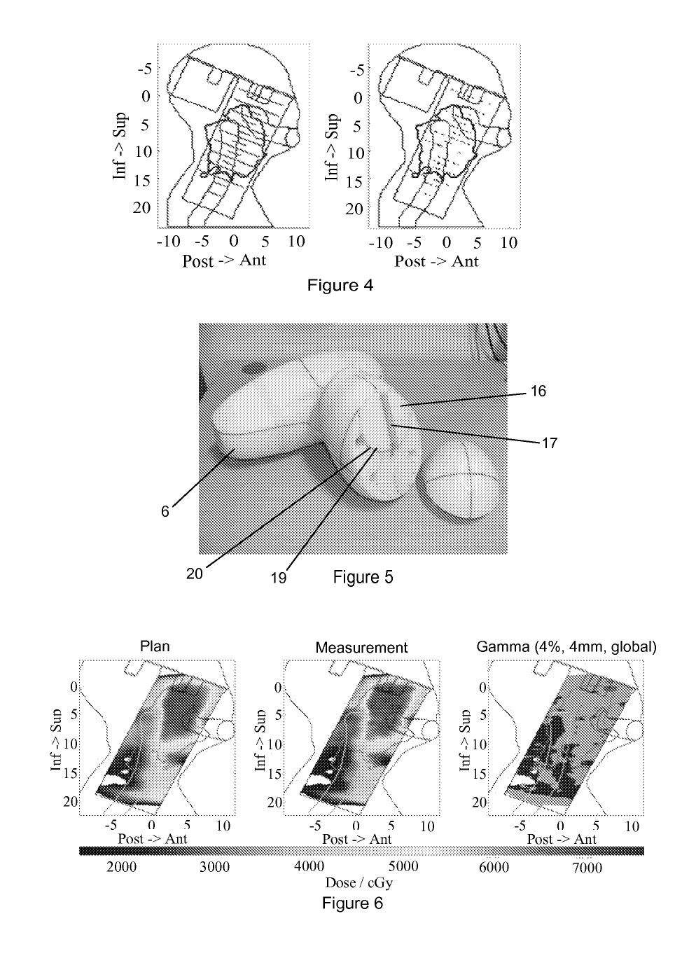

FIG. 4 shows a simulated use of a detector module containing several detector planes. Results of a simulated gamma analysis (3%, 3 mm) are shown for a typical oropharynx radiotherapy treatment plan. The PTV is outlined and failing points (γ>1) are shown dotted in the right-hand image;

FIG. 5 is a photograph of the prototype phantom of FIG. 1C with an alternative design of primary detector module received within a cavity defined by the phantom; and

FIG. 6 shows are colour coded images of a planned dose (left image), measured dose (middle image) and a comparison of the planned and measured doses (right image) mapped on to the geometry of a phantom according to the present invention using a radiosensitive 2D film detector.

phantom design.

A head and neck geometry modelling an average patient has been generated from CT datasets of 8 male and female patients lying in the treatment position. Average geometries were computed for the external body, the mandible, the sinuses and the spinal cord.

With reference to FIG. 1A, a model phantom was created within the TPS (Pinnacle v9.0) based on the average patient geometries, and a range of typical treatment plans (25 in total) was mapped on to the model to identify typical PTV locations. Based on this analysis, a space for receipt of detector module was defined within the phantom to provide maximum PTV coverage while also covering organs-at-risk, such as the spinal cord (see FIG. 1A). Referring to FIG. 1A, the space 1 for receipt of the detector module was defined in the phantom in such a way that the detector module would occupy a volume which included the spinal cord 2, common PTV locations 3 and very common PTV locations 4. An inhomogeneity representing the mandible 5 wraps around the detector module, such that measurements made within the detector module can test what impact the mandible 5 has on dose distribution.

Referring to FIG. 1B, phantom 6 defines two cylindrical cavities, one for receipt of a cylindrical primary detector module 7 and the other cavity for receipt of a cylindrical secondary detector module 8. Each module 7, 8 incorporates a plurality of slots for receipt of radiation detectors. The primary module 7 defines 15 slots arranged in an array which spirals inwards from the periphery of the module 7 to its centre (only 12 slots are visible in FIG. 1B because 3 slots are below the central circular cavity block). The secondary module 8 defines 4 slots arranged in a linear array extending from the periphery of the module 8 to its centre. The modules 7, 8 are designed for use with a PTW PinPoint™ionisation chamber (active volume 0.015 cm3), which can be slid into any one of the slots 9, 10 defined by either module 7, 8, thereby enabling absolute dose measurements to be made at almost any position within the detector module. A CNC-machined phantom prototype has been manufactured (see FIG. 1C) using ABS (ρ=1.05 gcm-3) as the phantom material. Homogeneous ABS air-cavity and mandible blocks are interchangeable with polyurethane foam (ρ=0.10 gcm-3) and glass-loaded polycarbonate (ρ=1.31 gcm-3) blocks to provide removable inhomogeneities.

Point Dose Measurements.

The PinPoint™ionisation chamber detector module provides a large number (>1800) of possible detector positions. Manual selection of appropriate detector locations for measurement in the PTV or spinal cord for comparison to TPS predictions can be complicated, due to the many points and the need to avoid steep dose gradients. Computer code has been written to automate the selection of optimum points in these regions (see FIG. 2).

Comparison Against TPS.

Preliminary evaluation of the prototype phantom has compared dose measurements within simple 10×10 cm square fields at various positions in the phantom against TPS predictions computed using bulk density overrides (see FIG. 3). Results indicate that applying a density override of 1.07 gcm-3 within the TPS provides good agreement with measured data.

Additional detector modules can be constructed for other dosimetry types, such as film, diode arrays or gel polymers, which will allow 2D or 3D dose distributions to be measured within the phantom according to the present invention. Methods of comparison between these experimentally measured distributions and those predicted by the planning system have been prepared using 3D gamma analysis techniques (see FIG. 4).

FIG. 5 is a photograph of the prototype phantom 6 shown in FIG. 1C but in which the primary detector module 7 shown in FIG. 1C has been replaced with an alternative design of primary detector module 16 which defines a rectangular slot extending across the diameter of the detector module 16 and into the detector module 16 for receipt of a sheet of X-ray sensitive film 17. The detector module 16 is shown partially mounted within its respective cavity defined by the phantom 6 such that the film 17 defines a plane in which lies the longitudinal axis of the simulated spinal cord of the phantom 6. The detector module 16 has been shaped such that at least a section of its outer surface 18 defines a series of straight edges 19. In this particular embodiment twelve straight edges are provided although any appropriate number may be employed, or they may be omitted completely such that that section of the outer surface 18 of the detector module 16 is smoothly curved. The straight edges 19 are provided to enable the detector module 16 to be securely received within its respective cavity in as many different orientations as there are straight edges by co-operating with complementary straight edges 20 defined by the phantom 6. Thus, in the specific embodiment depicted in FIG. 5, the detector module 16 and therefore the X-ray film 17 can be accurately and reproducibly mounted in any one of twelve predetermined orientations relative to the simulated anatomy of the phantom 6. It will be appreciated that this provides the user with a significant degree of flexibility in selected the best orientation of the X-ray film 17 to suit the proposed treatment regime.

Exemplary results obtained using the phantom 6 with the 2D X-ray film primary detector module 16 shown in FIG. 5 are presented in FIG. 6. The left hand picture is a colour coded image of a planned dose of radiotherapy mapped on to the geometry of the phantom 6. The middle picture is a colour coded image obtained from a 2D X-ray film mounted within the phantom 6. The right hand picture is an image which compares the planned (left hand image) and measured doses (middle image) using 3D gamma analysis. As can be seen, there is a high level of conformance between the planned and measured doses.

### CONCLUSION

An anatomically realistic head and neck phantom has been designed and constructed for use in the pre-treatment verification of IMRT and as an audit tool for centres conducting complex head and neck IMRT. The resulting system enables efficient and effective IMRT verification and audit in the head and neck, facilitating provision of this complex type of treatment.

It will be understood that numerous modifications can be made to the embodiments of the invention described above without departing from the underlying inventive concept and that these modifications are intended to be included within the scope of the invention. For example, the precise size and shape of the phantom housing may be adjusted to better suit a particular patient whose radiotherapy treatment plan is being verified using the phantom rather than the average model used above. While the detector modules used in the exemplary phantom were generally cylindrical in shape and defined a plurality of slots in spiral and linear arrangements for receipt of ionisation chamber detectors, it will be appreciated that the or each module to be used with the phantom may be of any appropriate size and shape, and may incorporate any suitable number, type and/or arrangement of spaces for receipt of radiation detectors. Moreover, while the exemplary phantom employed two cylindrical cavities for receipt of two different detector modules, any desirable number of cavities may be defined for receipt of any appropriate number of detector modules. Additionally, even though the results presented above are based upon a phantom designed for use within ionisation chamber radiation detectors, a significant advantage of the present invention results from its flexibility in being able to accommodate a wide range of different types of detector which may be used in separate treatment test cycles, or in combination, if desired.

What is claimed is:

1. A phantom for use in the auditing or verification of a proposed radiation therapy regime for administration to a patient, the phantom comprising:
a. a housing which is shaped to simulate the anatomical shape of a human head and neck; and
b. a radiation detector module configured to receive at least one radiation detector, wherein the housing defines a cavity in which the radiation detector module can be removeably received such that the radiation detector module occupies a predetermined location within the simulated head and neck of the housing, said predetermined location encompassing areas of the housing which simulate a target site to which it is proposed to administer radiation to the patient and a location of at least one organ that is susceptible to harm by administration of said radiation.

2. A phantom according to claim 1, wherein said organ is the spinal cord.

3. A phantom according to claim 1, wherein said predetermined location encompasses areas of the housing which simulate target sites that are commonly selected for the administration of radiation to treat a cancer selected from the group consisting of nasopharynx, oropharynx, hypopharynx, tongue, tonsil, thyroid and neck.

4. A phantom according to claim 1, wherein said cavity is dimensioned to encompass a majority of the volume of the simulated neck of the phantom.

5. A phantom according to claim 1, wherein said cavity is dimensioned to encompass areas of the simulated head of the phantom which simulate the pharynx and sinus cavities.

6. A phantom according to claim 1, wherein said cavity defines a cylinder that tapers linearly from a first end to an opposite second end.

7. A phantom according to claim 6, wherein a diameter of the first end is around 1 to 20% larger than a diameter of the second end.

8. A phantom according to claim 1, wherein said cavity possesses a longitudinal length that is greater than a diameter of its ends.

9. A phantom according to claim 1, wherein said cavity possesses a longitudinal length that is 50 to 250% greater than a diameter of its ends.

10. The phantom according to claim 1, wherein the cavity defines a longitudinal axis that is inclined relative to the horizontal when the phantom occupies a typical radiotherapy treatment position.

11. A phantom according to claim 1, wherein the cavity defined by the housing is adapted to receive one or more radiation detector modules supporting different types of radiation detectors.

12. A phantom according to claim 11, wherein each of said different types of radiation detector is selected from the group consisting of a radiosensitive gel, a radiosensitive film, a radiosensitive diode, an ionisation chamber, a thermoluminescent dosimeter, and a radioluminescent detector.

13. A phantom according to claim 1, wherein the cavity defined by the housing is adapted to receive one or more radiation detector modules supporting different numbers of radiation detectors.

14. A phantom according to claim 1, wherein the cavity defined by the housing is adapted to receive one or more radiation detector modules supporting different arrangements of radiation detectors.

15. A phantom according to claim 1, wherein said radiation detector module defines a plurality of locations for receipt of a radiation detector.

16. A phantom according to claim 15, wherein said plurality locations are provided in an arcuate path extending from a periphery of the radiation detector module to a centre of the radiation detector module.

17. A phantom according to claim 15, wherein said radiation detector is an ionisation chamber radiation detector.

18. A phantom according to claim 1, wherein said radiation detector defines a rectangular slot extending across a diameter of the detector module and into the detector module for receipt of a sheet of radiosensitive film.

19. A phantom according to claim 1, wherein an outer surface of said radiation detector module defines at least one straight edge to co-operate with a complementary straight edge defined by the phantom to locate the radiation detector in a predetermined orientation relative to the phantom.

20. A phantom according to claim 1, wherein the housing defines at least one cavity for receipt of a removable fixture at a location corresponding to that of a heterogeneity in the structure of at least one of the human head and the human neck.

21. A phantom according to claim 20, wherein said heterogeneity is an air cavity or a mandible.

22. A phantom according to claim 20, wherein said removable fixture is made of a different material to the remainder of the housing.

23. A method for auditing a radiotherapy regime using a phantom according to claim 1, the method comprising:
a. creating a radiotherapy treatment plan on a patient CT dataset;
b. transferring said radiotherapy treatment plan from the patient CT dataset on to a radiotherapy phantom CT dataset;
c. recalculating a dose distribution within the phantom as required to ensure that a location of a radiotherapy dose will lie in substantially the same region of the phantom CT dataset as in the patient CT dataset; and
d. exporting the radiotherapy treatment plan to a radiotherapy treatment machine for delivery to the phantom.

24. A method according to claim 23, further comprising exporting the radiotherapy treatment plan to a radiotherapy treatment machine for delivery to the patient.

25. A method for verifying a proposed radiotherapy regime using a phantom according to claim 1, the method comprising:
a. selecting one or more detector locations within the phantom to be used to measure a predetermined delivered dose of radiation;
b. providing the phantom in a treatment position;
c. inserting a detector or a plurality of detectors into the phantom so that they occupy said pre-selected detector location or locations;
d. providing required inhomogeneities within the phantom;
e. delivering said predetermined dose of radiation to the phantom;
f. measuring the dose of radiation delivered to the phantom using the one or more detectors;
g. comparing the measured dose of radiation to the predetermined dose of radiation; and
h. determining any differences between the measured dose of radiation and the predetermined dose of radiation.
